(19) United States
(12) Patent Application Publication
(10) Pub. No.: US 2013/0338639 A1
(43) Pub. Date: Dec. 19, 2013

(54) EAR WAX REMOVAL SYRINGE

(71) Applicant: Jacob Karpen, Brooklyn, NY (US)

(72) Inventor: Jacob Karpen, Brooklyn, NY (US)

(21) Appl. No.: 13/915,679

(22) Filed: Jun. 12, 2013

Related U.S. Application Data
(60) Provisional application No. 61/659,188, filed on Jun. 13, 2012.

(51) Int. Cl. A61M 3/02

(52) U.S. Cl. CPC A61M 3/0279
USPC 604/514; 604/218

(57) ABSTRACT

The invention is directed to a syringe used to irrigate the ear and remove earwax.

This application claims the benefit of U.S. Provisional Application No. 61/659,188, filed Jun. 13, 2012, which is incorporated by reference.

### BACKGROUND OF THE INVENTION

1. Field of the Invention

The invention is directed to a syringe used to irrigate an ear cavity and remove earwax.

2. Description of the Related Art

It is known to irrigate an ear canal with water or other fluid using a syringe, as disclosed in U.S. Pat. No. 3,651,808.

U.S. Pat. No. 6,706,023, which is incorporated by reference, discloses a tip for a syringe which can be used to irrigate an ear canal. The tip has a plurality of orifices that extend at an angle from a central bore, so that irrigation fluid is introduced into the ear canal at an angle and is not directed at the ear drum.

There continues to be a need for devices adapted for increased patient comfort which facilitate removal of cerumen (ear wax) from the irrigated ear canal.

### SUMMARY OF THE INVENTION

In one aspect, the invention is a syringe for irrigating an ear cavity and removing ear wax, comprising: a tubular syringe barrel having a distal end and a proximal end; a plunger at the proximal end positioned within the syringe barrel adapted to apply pressure to a fluid inside the syringe barrel and eject fluid from the distal end of the syringe; a bulbous tip on the distal end of the syringe barrel having a neck and a substantially semi-spherical bulbous portion wider than the neck located distally of the neck; laterally positioned fluid outlet portals in the bulbous tip; and a removable flexible porous cover on the bulbous tip.

A method of using the syringe to irrigate the ear canal and remove ear wax according to the invention comprises filling the syringe body with fluid, such as water; inserting the device into the patient's ear so that the bulbous tip forms a seal around the patient's outer ear canal; applying pressure to the syringe plunger to eject the fluid through the laterally positioned fluid portals to irrigate the ear canal; and swabbing the ear canal with the flexible porous cover to remove ear wax.

Those of ordinary skill in the art will readily be able to practice variants of the invention based on the description of preferred embodiments which follows. These embodiments are for the purposes of illustrating the scope of the invention and are not to be deemed limiting of the invention set forth in the appended claims.

### BRIEF DESCRIPTION OF THE DRAWINGS

FIG. 1 depicts an ear wax removal syringe having a flexible cover installed thereon.

FIG. 2 depicts a flexible cover which can be installed on the bulbous tip of the syringe.

### DETAILED DESCRIPTION OF THE INVENTION

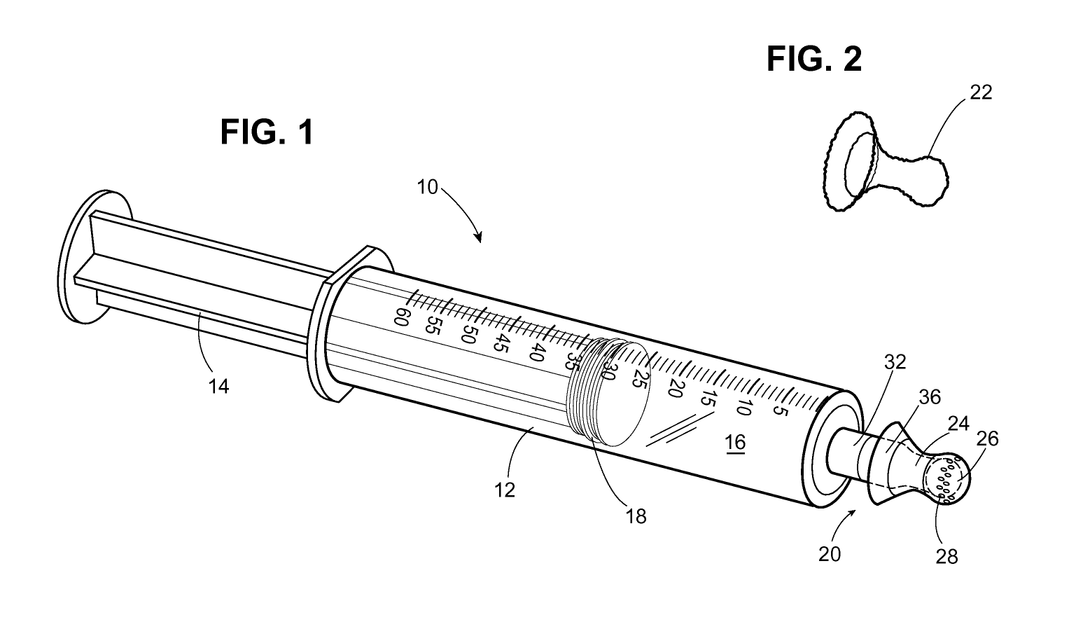

A device 10 for use cleaning an ear canal is depicted in FIG. 1, comprising a syringe body 12 and a plunger 14. Such syringes are known in the art and may be made by injection molding a suitable plastic as known in the art. Plunger 14 is adapted with a stopper 18 to apply pressure to a fluid in the cavity 16 of the syringe barrel and eject fluid from the distal end portion 20 of the syringe as the plunger is pushed through the syringe body 12. The fluid may be water or other medically appropriate fluid for cleaning an ear canal of cerumen (ear wax).

On the distal end portion 20 of the device, a bulbous tip 26 is adapted to fit inside the user's ear creating a seal that substantially prevents passage of fluid out of the user's ear past the bulbous tip. The bulbous tip 26 is an integral part of the distal end portion 20 extending from the syringe body, comprising a semi-spherical portion which tapers to a narrow neck portion 24. The portion of the bulbous tip 26 contacting the user's ear, is made of a pliable hypoallergenic material which can be compressed, such as elastomeric foam, rubber, silicone, vinyl or acrylic polymers, dimensioned so that the distal end of the tip 26 may be placed into the external ear canal but portions of the tip 26 nearer the proximal end of the tip will come into sealing contact with the patient's external ear canal 26. Because the tip is somewhat pliable, it will conform to the shape of the patient's external ear canal.

In embodiments, the distal end portion 20 comprises a connecting portion 32 which forms a unitary structure with the syringe body. Connecting portion 32 may be molded in one piece with the syringe body by injection molding or other technique. In FIG. 1, the connecting portion 32 is a cylinder integral with the distal end of syringe body 12. The connecting portion 32 is tubular and allows passage of fluid to the bulbous tip 26. The open distal end of the connecting portion 32 may be provided with threads or snap-fit features that permit the bulbous tip 26 to be securely attached to the syringe body 12. Alternatively, adhesives, welding, or equivalent means for secure attachment may be used. In embodiments, bulbous tip 26 is attached to the connecting portion 32 by an adapter 36, which is intermediate the bulbous tip and the connecting portion 36. Mating features are provided on the adapter to allow for fluid communication between the bulbous tip 26 and the syringe body 12. In one embodiment, the device 10 is sold with a single bulbous tip 26 attached to the syringe body 12, which is not intended to be removed by the user. In another embodiment, the bulbous tip 26 may be removable such that a different sized bulbous tip 26 may be attached.

The device 10 comprises between 2 and 20, preferably between 2 and 10 laterally positioned fluid outlet portals 28 on the bulbous tip 26. In order to avoid excessive pressure of fluid exiting the bulbous tip 26 and entering the ear, which could damage the ear, the portals are disposed around the side of the bulbous tip, forming an angle to the direction of the opening of the ear canal. It will be understood that the bulbous tip is not provided with a portal in the axial direction aligned with the opening of the ear canal. Rather, each fluid outlet portal 28 preferably forms an angle with the longitudinal axis of the syringe. That is, preferably the fluid outlet portals 28 are forward of a radial axis, so that fluid is directed toward the sealed part of the ear canal but not directly into it. In one embodiment, two fluid outlet portals are disposed on opposite sides of the bulbous tip for passage of fluid from the syringe into the patient's ear. In alternative embodiments, the device has a plurality of portals which extend from a central or distributed base near the syringe to various points at the tip of the syringe. This embodiment allows fluid to exit the bulbous tip 26 at a plurality of angles. In still another embodiment, a plurality of fluid outlet portals 28 is present, each spaced equidistantly from the other and spaced between the tip of the device and the radial axis. The size of the fluid outlet portal may vary depending on the number of outlet portals and the size of the syringe, typically in a range of about 0.01 mm to about 1 mm in diameter.

FIG. 2 depicts a flexible porous cover 22 which can be used to cover the bulbous tip 26, as shown in FIG. 1 to prevent the transmission of dirt, bacteria, and viruses from one user to another. Instead of having to replace the bulbous tip with each use, the user may simply replace a flexible porous cover. A plurality of flexible porous covers 22 may be provided with a single syringe as a kit. The flexible porous cover may be foam, sponge, or preferably sterilized cotton.

The flexible porous cover 22 preferably comprises sterilized cotton, such as used in a bandage, i.e., a mesh, felt or knit gauze cotton material. The cover 22 should be thin so that the cover 22 conforms to the bulbous tip 26 and the fluid readily penetrates the porous material. As the semi-spherical region of the bulbous tip partially enters the ear, the wide part acts as a stopper, preventing further entry into the ear and sealing the inside of the ear with the tip. The cover material is pressed against the ear and forms a better seal around the ear cavity. Typically, the thickness of the cover is in a range of about 0.5 to about 2.0 mm, although this dimension is not critical and may be adapted according to necessity.

Preferably, the flexible porous cover 22 is shaped so as not to be too easily removed from the bulbous tip 26 after being installed. For example, the cotton material may be provided in an elastic knit with an opening that stretches around the bulbous tip 26 and grasps neck portion 24. In this way, the cover material can be used as a cotton swab after the ear canal is irrigated. Cotton material may also be soaked in a medicated solution, press-formed and dried in a shape that slides tightly over the bulbous tip 26. Other equivalent means of making a flexible porous cover material that conforms to the shape of the bulbous tip without sliding off during use will be apparent to those of ordinary skill in the art.

A method of using the syringe to irrigate the ear canal and remove ear wax according to the invention comprises first filling the syringe body with fluid. As noted above, the fluid may be water, or some medically appropriate fluid for irrigating the ear. The fluid may be poured into the syringe body prior to installing the plunger, or fluid may be aspirated into the syringe by inserting the distal end 20 of the syringe in the fluid and pulling the plunger. Thereafter, the method comprises inserting the device into the patient's ear so that the bulbous tip forms a seal around the patient's outer ear canal. The presence of flexible porous material on the outside of the bulbous portion facilitates fitting the tip against the ear cavity and forming a seal. Applying pressure to the syringe plunger ejects the fluid through the laterally positioned fluid portals to irrigate the ear canal. The user then swabs the ear canal with the flexible porous cover to remove ear wax. The user can also swab the ear canal with the device without first irrigating.

The above description of the preferred embodiments is not to be deemed to limit the invention, which is defined by the following claims.

1. A syringe for irrigating an ear cavity and removing ear wax, comprising:
a tubular syringe barrel having a distal end and a proximal end;
a plunger at the proximal end positioned within the syringe barrel adapted to apply pressure to a fluid inside the syringe barrel and eject fluid from the distal end of the syringe;
a bulbous tip on the distal end of the syringe barrel having a neck and a substantially semi-spherical bulbous portion wider than the neck located distally of the neck;
laterally positioned fluid outlet portals in the bulbous tip; and
a removable flexible porous cover on the bulbous tip.

2. The syringe according to claim 1, comprising between 2 and 10 laterally positioned fluid outlet portals, each forming an angle with a longitudinal axis of the syringe.

3. The syringe according to claim 1, wherein the flexible porous cover comprises sterilized cotton.

4. The syringe according to claim 1, wherein the flexible porous cover is elastic and shaped to stretch over the bulbous tip and grasp the neck of the bulbous tip.

5. The syringe according to claim 1, comprising a cylindrical connecting portion on the distal end of the syringe body formed integrally with the syringe body having an opening and features for secure attachment to the bulbous tip; and
a cylindrical adapter on the proximal end of the bulbous tip having an opening on the distal end thereof mating with features on the distal end of the cylindrical connecting portion.

6. The syringe according to claim 5, wherein the bulbous tip is removable from the syringe body by the user.

7. A kit comprising a syringe according to claim 1 and a plurality of flexible porous covers.

8. A method of using a syringe according to claim 1 to irrigate the ear canal and remove ear wax, comprising:
providing a syringe according to claim 1;
filling the syringe body with fluid;
inserting the device into a patient's ear and forming a seal with the bulbous tip against the patient's outer ear cavity;
applying pressure to the syringe plunger to eject the fluid through the laterally positioned fluid portals to irrigate the ear canal; and
swabbing the ear canal with the flexible porous cover to remove ear wax.
